(19) United States
(12) Patent Application Publication
(10) Pub. No.: US 2012/0172408 A1
(43) Pub. Date: Jul. 5, 2012

(54) ACTIVE INGREDIENT COMPRISING INDOCYANINE GREEN AND/OR INFRACYANINE GREEN

(75) Inventor: Reinhardt Thyzel, Eckental (DE)

(73) Assignee: A.R.C. LASER GMBH, Nuernberg (DE)

(21) Appl. No.: 13/181,295

(22) Filed: Jul. 12, 2011

(30) Foreign Application Priority Data
Jan. 3, 2011 (DE) 10 2011 000 020.8

(51) Int. Cl. A61K 31/403; A61P 31/00; A61P 29/00; C07D 403/06

(52) U.S. Cl. 514/411; 548/427

(57) ABSTRACT

The present invention relates to an active ingredient including indocyanine green and/or infracyanine green for use in treating dental or periodontal infectious diseases under irradiation with light, more particularly laser light, from a wavelength range between 790 nm and 830 nm.

### CROSS-REFERENCE TO RELATED APPLICATIONS

The present invention claims the benefit of priority to German Patent Application NO. DE 10 2011 000 020.8, filed Jan. 3, 2011, entitled “Active Ingredient Comprising Indocyanine Green and/or Infracyanine Green,” the entire content of which is incorporated by reference herein.

### BACKGROUND OF THE INVENTION

1. The Field of the Invention

The invention relates to an active ingredient comprising indocyanine green and/or infracyanine green.

2. Background and Relevant Art

Indocyanine green (ICG) is, according to www.wikipedia.de, a fluorescent dye which is used in medicine as an indicator in heart, vascular, liver and eye diseases. It is administered intravenously and, depending on the activity of the liver, eliminated from the body with a half life of about 3-4 minutes. ICG sodium salt is normally available in powder form and can be dissolved in different solvents; 5% (<5% depending on the batch) sodium iodide is usually added for better solubility.

For cases of iodine intolerance, an iodide-free indocyanine green is also known, which is referred to as infracyanine green (IfCG or IFC green).

Furthermore, according to www.wikipedia.de, there are initial studies in dermatology which indicate that under certain circumstances—such as appropriate laser intensity and certain wavelengths—ICG is able to trigger a photodynamic reaction and thus might be used in the medium term as a PDT therapeutic as well. Concerning this, there are initial studies on dermatological use by Christoph Abels, Sonja Fickweiler, Petra Weiderer, Wolfgang Bäumler, Ferdinand Hofstädter, Michael Landthaler and R.-M. Szeimies: Indocyanine green (ICG) and laser irradiation induce photooxidation. In: Archives of Dermatological Research. Springer Berlin/Heidelberg, Vol. 292, Number 8, August 2000, and also in R.-M. Szeimies, T. Lorenzen, S. Karrer, C. Abels and A. Plettenberg: Photochemotherapie kutaner Aids-assoziierter Kaposi-Sarkome mit Indocyaningrün and Laserlicht [Photochemotherapy of cutaneous Aids-associated Kaposi's sarcoma with indocyanine green and laser light]. In: Der Hautarzt [The Dermatologist], Springer, Berlin/Heidelberg, Vol. 52, Number 4, March 2001, pages 322-326.

The absorption spectrum and fluorescence spectrum of ICG are in the near-infrared range. Both are strongly dependent on the solvent used and on the concentration. ICG absorbs mainly between 600 nm and 900 nm and emits fluorescence between 750 nm and 950 nm. The large overlap of the absorption and fluorescence spectra leads to strong re-absorption of the fluorescence by ICG itself

The fluorescence spectrum is quite broad. Its maxima are at about 810 nm in water and at about 830 nm in blood. For medical applications which are based on absorption, the absorption maximum at about 800 nm (in blood plasma at small concentrations) is important. In combination with fluorescence detection, use is often also made of lasers of a wavelength of around 780 nm. At this wavelength, ICG still absorbs very well, and it is nevertheless still technically possible to suppress the excitation light in order to detect the fluorescence.

In dentistry, a problem in the treatment of gingiva and gingival pockets is that bacteria can reach the bloodstream and, as a result, can lead to serious, occasionally fatal, infections. Therefore, use is concomitantly made here of antibiotics which are administered orally.

In the company brochure “Die Antimikrobielle Photodynamische Therapie (PDT)” [“Antimicrobial Photodynamic Therapy (PDT)”] by Dr. Mettraux and Professor Dr. Dörtbudak from MS Dental, antimicrobial photodynamic therapy under the trade name HELBO for treating periodontitis marginalis, peri-implantitis and cavities and also for endodontic therapy is offered by the active photosensitizer ingredient HELBO BLUE, which is based on methylene blue, in combination with a laser having a 670 nm wavelength and 75 mW of power and also with an optical fiber whose fiber tip radiates the red light radially with uniform light distribution by means of a microlens surrounding the tip. The gingiva to be treated is wetted with the active ingredient HELBO BLUE and then irradiated with the laser light with the light conductor end held above into the mouth, whereby bacterial germs are killed.

### BRIEF SUMMARY OF THE INVENTION

Embodiments of the present invention are based on the knowledge obtained from experiments that ICG has an outstanding effect against microscopic organisms, more particularly an antimicrobial effect, for treating infections on gingiva and on teeth under irradiation with light, more particularly laser light, having a wavelength in the range from 780 to 830 nm.

According to Claim 1, there is therefore proposed an active ingredient comprising indocyanine green (ICG) and/or infracyanine green (IfCG) for use in treating dental or periodontal infectious diseases under irradiation with light, more particularly laser light, from a wavelength range between 790 nm and 830 nm, more particularly between 800 nm and 820 nm. In other words, the active ingredient is used for photodynamic therapy on gingiva in the mouth of a patient in order to control bacteria and other microorganisms there, wherein the effect of the ICG or IfCG against the bacteria, fungi or other microorganisms is induced by irradiation with light, more particularly laser light, from the wavelength spectrum stated. Thus, there is claimed according to the invention a further medical indication for the active ingredient ICG and a first medical indication for the active ingredient IfCG.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### DETAILED DESCRIPTION OF THE PREFERRED EMBODIMENTS

The active ingredient preferably comprises at least one solvent, generally water, more particularly water low in anions, for dissolving ICG or IfCG, with the concentration of ICG and/or IfCG in the solvent being at least 250 μg based on 1 ml of solvent. This comparatively high concentration has the advantage of compensating for dilution of the active ingredient in the oral cavity owing to saliva. Preferably, the concentration of ICG or IfCG is chosen to be even higher and is then at least 500 μg, but generally not more than 5000 μg, based on 1 ml of solvent.

Specific and advantageous uses of the active ingredient are intended in wound disinfection during tooth extractions or in the endodontic treatment or the treatment of infections in dental root canals or in the treatment of peri-implantitis.

Especially advantageous is the use of the active ingredient in the treatment of infections on gingiva or for controlling bacteria and/or fungi on gingiva, more particularly when cleaning gingival pockets or gaps adjacent to the gingiva between the teeth and gingiva.

When using the active ingredient, the light, more particularly laser light, has a continuous output or irradiance greater than 100 mW, preferably from a range of 100 mW to 200 mW and/or for at least 30 s, and/or an energy input or an energy density greater than 30 J/cm², preferably between 30 J/cm² and 60 J/cm².

In an especially advantageous further development, there is proposed for the use of the active ingredient a system for treating dental or periodontal infectious diseases, which system comprises at least one active ingredient applicator for applying the active ingredient according to the invention and at least one light applicator for irradiating the applied active ingredient with light, more particularly laser light, from a wavelength range between 790 nm and 830 nm, more particularly 800 nm to 820 nm.

The system preferably comprises a light source, more particularly a laser light source, which, during the treatment, preferably generates a continuous output or irradiance greater than 100 mW, preferably from a range of 100 mW to 200 mW and/or for at least 30 s, and/or delivers an energy input or an energy density greater than 30 J/cm², preferably between 30 J/cm² and 60 J/cm².

More particularly, the system also acts to detach or separate or necrotize inflamed gingival tissue in which the active ingredient is present.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

I claim:

1. An active ingredient comprising:
indocyanine green and/or infracyanine green for use in treating dental or periodontal infectious diseases under irradiation with light, more particularly laser light, from a wavelength range between 790 nm and 830 nm.

2. The active ingredient according to claim 1 comprising:
at least one solvent, more particularly water, more particularly water low in anions;
wherein the concentration of the indocyanine green and/or infra-cyanine green in the solvent is at least 250 μg, preferably at least 500 μg, based on 1 ml of solvent, more particularly not more than 5000 μg based on 1 ml of solvent.

3. The active ingredient according to claim 1, for use in wound disinfection during tooth extraction.

4. The active ingredient according to claim 1, for use in the endodontic treatment or the treatment of infections in dental root canals.

5. The active ingredient according to claim 1, for use in the treatment of peri-implantitis.

6. The active ingredient according to claim 1, for use in the treatment of infections on gingiva or for controlling bacteria and/or fungi on gingiva, more particularly when cleaning gingival pockets or gaps adjacent to the gingiva between the teeth and gingiva.

7. The active ingredient according to claim 1, wherein the light intended for irradiation, more particularly laser light, has a continuous output or irradiance greater than 100 mW, preferably from a range of 100 mW to 200 mW and/or for at least 30 s, and/or an energy input or an energy density greater than 30 J/cm², preferably between 30 J/cm² and 60 J/cm².

8. A system for treating dental or periodontal infectious diseases with at least one active ingredient applicator for applying the active ingredient comprising:
at least one light applicator for irradiating the applied active ingredient with light, more particularly laser light, from a wavelength range between 790 nm and 830 nm.

9. The system according to claim 8, comprising:
a light source, more particularly a laser light source, which, during the treatment, generates a continuous output or irradiance greater than 100 mW, preferably from a range of 100 mW to 200 mW and/or for at least 30 s, and/or delivers an energy input or an energy density greater than 30 J/cm², preferably between 30 J/cm² and 60 J/cm²;
wherein the light applicator is coupled or can be coupled to the light source.

10. The system according to claim 8, for detaching or separating or necrotizing inflamed gingival tissue in which the active ingredient is present.
